(19) United States
(12) Patent Application Publication
(10) Pub. No.: US 2025/0269238 A1
(43) Pub. Date: Aug. 28, 2025

(54) REHABILITATION TRAINING SYSTEM AND METHOD

(71) Applicants: Akashdeep RANDHAWA, Robbinsville, NJ (US); Shaun RAHIMI, Santa Clara, CA (US)

(72) Inventors: Akashdeep RANDHAWA, Robbinsville, NJ (US); Shaun RAHIMI, Santa Clara, CA (US)

(21) Appl. No.: 19/052,595

(22) Filed: Feb. 13, 2025

Related U.S. Application Data
(60) Provisional application No. 63/558,894, filed on Feb. 28, 2024.

(51) Int. Cl. A63B 24/00; A61B 5/00; A61B 5/397; A63B 71/06

(52) U.S. Cl. CPC A63B 24/0075; A61B 5/397; A61B 5/486; A63B 71/0622; A61B 2505/09; A63B 2071/0625; A63B 2071/0652; A63B 2071/0655

(57) ABSTRACT

Methods and apparatus for rehabilitation training are disclosed herein. In one variation of the invention, a system for rehabilitation training is provided, comprising a sensor configured to capture one or more EMG signals from a muscle group of a user performing one or more physical movements, wherein the sensor device is coupled to the user, a processor configured to analyze the one or more EMG signals, wherein the processor is configured to determine a symmetry score, wherein the symmetry score is based on muscle activation in the muscle group, and a display configured to provide guidance to the user during the one or more physical movements, wherein the display comprises a dynamic feedback for the user based on the symmetry score.

### CROSS-REFERENCE TO RELATED APPLICATIONS

This application claims priority to U.S. Provisional Application No. 63/558,894 filed Feb. 28, 2024, which is herein incorporated by reference to the same extent as if each such individual publication or patent application were specifically and individually indicated to be so incorporated by reference.

### FIELD OF THE INVENTION

The present apparatus and methods relate generally to rehabilitation training machines, and more particularly to an adaptive rehabilitation training machine with surface electromyography biofeedback.

### BACKGROUND OF THE INVENTION

In helping patients recover from musculoskeletal (MSK) injury or disease, physical/occupational therapists and patients currently use antiquated, subjective technology to track progress during treatment of MSK conditions that lack engagement.

Studies have shown that patients showed higher improvements in muscle strength and functions when rehabilitated with Surface Electromyography BioFeedback (sEMG BF) combined with conventional physiotherapy compared to those who were only treated with conventional physiotherapy. Currently, there are several larger surface electromyography (EMG) systems which are much larger and require therapists to manually analyze data to set goals for patients. This technology also requires a significantly longer period to set patients up with electrodes and can inhibit certain patient movements for desired exercises.

Computer vision (via smartphone cameras) could be used to provide real-time biofeedback guidance for physical therapy. This could be done by measuring the range of motion performed by the patient and determining if a movement is successfully performed. This approach does not have sensors to attach to the patient. The disadvantage of this approach includes a lack of physiological measurements to determine strength and fatigue, no transcutaneous electrical nerve stimulation (TENS) machine to promote muscle strength and decrease pain, and a lack of computer vision which cannot track all isokinetic movements of the body to determine muscular performance.

In view of the above shortcomings in the current technology, there is a long-felt need for a product that provides objective data to showcase patient recovery and provides live guided feedback on performance of exercises through automated calibration system and TENS machine. Such methods and devices can engage patients and allow therapists to take a more calibrated approach to patient recovery.

### SUMMARY

In one variation of the invention, a system for rehabilitation training for a user is disclosed. The system can comprise a sensor configured to capture one or more EMG signals from a muscle group of the user when performing one or more physical movements; a processor configured to analyze the one or more EMG signals, wherein the processor is configured to determine a symmetry score which is indicative of a relative symmetry of the muscle group during the one or more physical movements and is based on a comparison of muscle activation in the muscle group, wherein the processor is configured to establish an adaptive target goal based on the muscle activation in the muscle group; and a display configured to provide guidance to the user during the one or more physical movements, wherein the display comprises a dynamic feedback for the user based on the symmetry score, wherein the guidance comprises a dynamic feedback for the user based on the symmetry score and is auditory, haptic, and/or visual to guide the user on a target muscle activation level.

The processor can be configured to calculate the symmetry score across one or more phases of muscle activation. The processor can be configured to identify variations in one or more muscle activation patterns that influence movement precision and control.

The processor can be configured to establish a calibrated goal based on a predetermined threshold relating to the muscle activation, wherein the predetermined threshold is a percentage of the highest value of the one or more EMG signals. The guidance can be auditory, haptic, and/or visual.

The processor can calculate the symmetry score by taking an average asymmetry of muscle activation via the one or more EMG signals between a first lateral side of the user and a second lateral side of the user. The processor can calculate the symmetry score in real-time during the one or more physical movements by comparing muscle activation via the one or more EMG signals between a first lateral side of the user and a second lateral side of the user. The processor can calculate the symmetry score in a plurality of phases of the one or more physical movements. The symmetry score can comprise a consistency of muscle activation via the one or more EMG signals during the one or more physical movements, wherein the processor is configured to assess the consistency by capturing variability and neuromuscular control trends of the muscle group over. The symmetry score can comprise stability and endurance trends to measure fatigue of the muscle group during the one or more physical movements.

The sensor can comprise a magnetic or mechanical attachment to a conductive garment. The sensor can comprise a magnetic or mechanical attachment to a hydrogel electrode applied to the user.

In one variation of the invention, a method for measuring rehabilitative exercises of a patient is disclosed. The method can comprise applying a sensor to a user, capturing one or more EMG signals with the sensor, wherein the one or more EMG signals are derived from a muscle group of a user when performing one or more physical movements; analyzing the one or more EMG signals with a processor; determining a symmetry score with the processor, wherein the symmetry score which is indicative of a relative symmetry of the muscle group during the one or more physical movements and is based on a comparison of muscle activation in the muscle group, wherein the processor is configured to establish an adaptive target goal based on the muscle activation in the muscle group; and providing guidance to the user with a display during the one or more physical movements, wherein the display comprises a dynamic feedback for the user based on the symmetry score and is auditory, haptic, and/or visual to guide the user on a target muscle activation level.

The method can further comprise calculating the symmetry score by taking an average asymmetry of muscle activation via the one or more EMG signals between a first lateral side of the user and a second lateral side of the user. The method can further comprise calculating the symmetry score in real-time during the one or more physical movements by comparing muscle activation via the one or more EMG signals between a first lateral side of the user and a second lateral side of the user. The method can further comprise calculating the symmetry score in a plurality of phases of the one or more physical movements. The method can further comprise displaying a prompt to the user based on the symmetry score.

In some variations, the method can comprise determining a calibrated goal based on a predetermined threshold relating to the muscle activation.

### BRIEF DESCRIPTION OF THE DRAWINGS

The drawings constitute a part of this specification and include exemplary embodiments of the invention, which may be embodied in various forms. It is to be understood that in some instances various aspects of the embodiments may be shown exaggerated or enlarged to facilitate an understanding of the embodiments.

FIG. 1A illustrates a system for rehabilitation training in accordance with one variation of the present invention.

FIG. 1B illustrates the placement of EMG sensors on a user in accordance with one variation of the present invention.

FIGS. 2A to 2F illustrate various interfaces for use with the display in accordance with one variation of the present invention.

FIG. 3 illustrates a surface EMG flowchart showing how the elements are connected according to one variation of the present invention.

FIG. 4 illustrates a TENS plus EMG flowchart showing how the elements are connected according to one variation of the present invention.

### DETAILED DESCRIPTION

The description of illustrative embodiments according to principles of the present disclosure is intended to be read in connection with the accompanying drawings, which are to be considered part of the entire written description. In the description of embodiments of the disclosure disclosed herein, any reference to direction or orientation is merely intended for convenience of description and is not intended in any way to limit the scope of the present disclosure. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the disclosure are illustrated by reference to the exemplified embodiments. Accordingly, the disclosure expressly should not be limited to such exemplary embodiments illustrating some possible non-limiting combination of features that may exist alone or in other combinations of features; the scope of the disclosure being defined by the claims appended hereto.

This disclosure describes the best mode or modes of practicing the disclosure as presently contemplated. This description is not intended to be understood in a limiting sense, but provides an example of the disclosure presented solely for illustrative purposes by reference to the accompanying drawings to advise one of ordinary skill in the art of the advantages and construction of the disclosure. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

Surface Electromyography BioFeedback (sEMG BF) is known to be a valid and reliable indicator of estimating muscle recruitment and uses neurosignals to quantify muscle tension. EMG parameters, such as EMG amplitude, help to understand the recruitment characteristics of the muscle of interest. As compared to other sEMG BF devices, a device according to an embodiment the device is significantly smaller in size (e.g., as small as the size of a quarter) and wireless, allowing it to be attached to any muscle of interest.

Both the device and software are designed with one objective of intensifying the case of use. For example, a user using the device can access the interface within, e.g., two minutes, with the aim of optimizing the use while offering maximum functionality and respecting business limitations. As the muscle contracts, the system according to one embodiment quantifies contraction of the corresponding muscle, and if a threshold value is met for contraction, an auditory and/or visual alert may be generated. Patients can contract a muscle sufficiently to generate the auditory/visual feedback. The threshold may be based on the initial warm up exercise, which becomes the target for a patient to achieve during subsequent exercises. An auditory sound and/or visual cue may be generated with connectivity to a provider's and/or patient's remote device such as their smart phones and/or smart devices. For objective data, the average amplitude may be calculated during the entire study that takes into consideration both time and magnitude in contrast to peak amplitude, providing measurement of muscle strength in patients. In one embodiment, a final performance score may be provided based on percentage of completed repetitions and recorded EMG intensity on a scale from 0-100. In one example, the final performance score may be calculated using the following formula:

Score=(average raw value+[(percent of successful exercises−70)×15])/7.

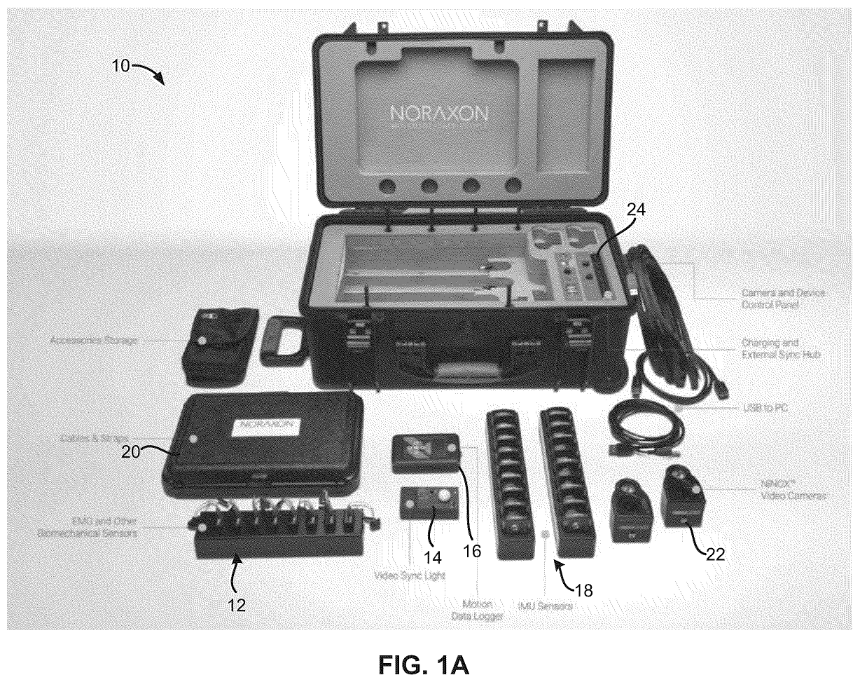

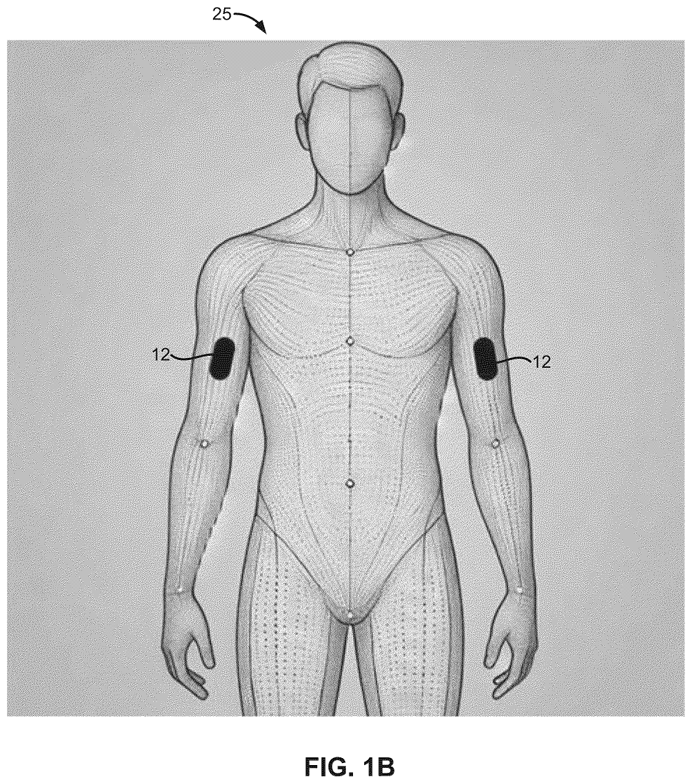

FIG. 1 illustrates one example of a system for rehabilitation training. The system 10 can comprise one or more surface EMG sensors 12, a video sync light 14 a motion data logger 16 to capture raw data of the user's movement over time, one or more inertial measurement unit (IMU) sensors 18, one or more cables and strips 20 for wired connections to a computer, a video camera 22 for recording the user's movements for subsequent review, and a control panel 24 having a control unit within. In some variations, the system 10 can include a device (e.g., a mobile device such as a smartphone, computer, tablet, etc.) in communication with the various components such as the EMG sensors 12, IMU sensors 18, motion data logger 16, etc. for processing the data (as described herein) and which may also include a monitor or display for displaying the results and data to the user (e.g., symmetry information, etc.) in real time.

The one or more EMG sensors 12 can comprise a magnetic and/or mechanical attachment to a conductive garment to couple to the user. In some variations, the one or more EMG sensors 12 can comprise a magnetic and/or mechanical attachment to a hydrogel electrode applied to the user or any other EMG sensor which can be attached or otherwise secured directly to the user or to a garment or strap worn by the user.

The one or more EMG sensors 12 can capture one or more EMG signals from a muscle group of a user performing one or more physical movements, or an exercise. The muscle group can be selected from the group of, not limited to: shoulder muscles, arm muscles, back muscles, chest muscles, abdominal muscles, hip muscles, thigh muscles, and leg muscles, or any other muscle group. The one or more EMG sensors 12 can detect the EMG signals from muscle activation when the muscles contract and can subsequently transmit the data to the control unit for processing either through a wired or wireless communication protocol.

In some variations, the one or more EMG sensors 12 can be wirelessly coupled to the control panel. In other variations, the one or more EMG sensors 12 can be coupled via wires to the control panel.

The control unit can comprise a processor, controller, or other computing unit configured to analyze the one or more EMG signals in real-time. To this end, the processor can be configured to determine a numerical symmetry score (as described in detail herein) and other metrics based on a comparison of muscle activation in the muscle group. The symmetry score can be generated and displayed to the user as an overall assessment of their progress during their exercise or a real-time score during the exercise to provide guidance or feedback on asymmetry in individual exercise repetitions.

The processor can be configured to establish an adaptive target goal or baseline during the one or more exercise. The adaptive target goal can be established by the processor based on the muscle activation in the muscle group. For example, in response to the EMG signals, the processor can calibrate a goal based on a predetermined threshold relating to the muscle activation. The predetermined threshold can a percentage of the highest value of the one or more EMG signals (e.g., 80% of the highest EMG signal recorded). It should be noted that the processor can establish the adaptive target goal during the one or more exercises, for example, as peak muscle activation changes.

The one or more IMU sensors 18 can be configured to measure motion in order to access EMG activation level at different points of specified biomechanical movements. For example, the one or more IMU sensors 18, in addition to the EMG sensors, can be placed on the user, for example, on a calf during a propulsion phase, or on a foot to measure gait and ground reaction force.

FIG. 1B illustrates the placement of the one or more EMG sensors 12 on a user 25 in accordance with one variation of the present invention. As the symmetric activation of the muscles of the user are desirably sensed for monitoring and display, the EMG sensors 12 may be positioned along any number of muscle groups so that muscle groups are monitored on both the corresponding left and right side of the patient's body. In the variation shown in FIG. 1B, the one or more EMG sensors 12 can be attached to the user 25 at the user's left and right biceps. The one or more exercises in this example can thus be targeted towards measuring activation, e.g., of the biceps, and/or nearby muscles (e.g., triceps) to generate the symmetry score. In some variations, a plurality of EMG sensors 12 can be positioned on each side of the user's anatomy (e.g., two sensors can be placed on each of the left side and the right side).

It should be understood that the one or more EMG sensors 12 can be placed to target other muscle groups of the user 25, including, but not limited to: shoulder muscles (e.g., deltoids, rotator cuff), forearm muscles, chest muscles (e.g., pectorals), upper back and scapular muscles (e.g., trapezius, rhomboids), abdominal muscles (e.g., rectus abdominis, obliques), lower back muscles (e.g., erector spinae), hip muscles (e.g., gluteus maximus, hip flexors), thigh muscles (e.g., quadriceps, hamstrings, adductors), lower leg muscles (e.g., calves, tibialis anterior), and foot muscles (e.g., plantar muscles, anterior muscles). It should also be understood that one or more IMU sensors 18 may be placed at any of the aforementioned locations.

With the EMG sensors 12 attached to the predetermined corresponding muscle groups (e.g., left and right muscle groups), the user may view the sensed and processed data in real time upon a display during, e.g., a therapy session. This information may also be stored and/or transmitted to a third party which may be located remotely or in proximity to the user.

The symmetry score can be derived from three separate parts: Part One is the average symmetry for the overall exercise, Part Two is a weighted score for contributions of the left and right sides of the body to asymmetry calculations, and Part Three is a phase symmetry score and standard deviation (e.g., for different stages of the workout such as beginning, middle, and end).

Part One calculates the average symmetry score of muscle activation between the left and the right sides for the entire exercise. The average symmetry score is calculated according to the following equation:

$$Symmetry Score ( Average Asymmetry ) = \frac{{\sum }_{i = 1}^{n} \vert Symmetry {Value}_{i} \vert}{n}$$

where: Symmetry Value; is the symmetry measurement for a single trial (range: −100 to 100), and n is the total number of measurements.

The average symmetry score output by this equation can range from, e.g., 0 to 100, where 0 is complete asymmetry and 100 is perfect symmetry. This score can be displayed to the user and/or physician after the exercise is complete. The score can be categorized into multiple ranges which may also be color-coded for the display.

In one example, an average symmetry score range between 95 and 100 can be displayed with a green color, indicating exceptional symmetry (i.e., well-balanced activation between the left and right sides of the user). An average symmetry score range between 85 and 95 can be displayed with a yellow color, indicating minor asymmetry (i.e., a slight dominance of one side compared to the other side). An average symmetry score between 0 and 85 can be displayed with a red color, indicating significant asymmetry (i.e., major dominance of one side which can lead to joint or muscular issues if not addressed).

The average symmetry score can provide the user with feedback regarding balance of muscle activation of their overall exercise. If the user has significant asymmetry of muscle activation between their left side and right side (e.g., first lateral side and second lateral side), the symmetry score will be relatively lower than compared to higher symmetry between their left side and right side. In this case, long-term joint and/or muscular issues can arise if the asymmetry is not addressed. The user may receive a display message that recommends targeting strengthening and improving technique accordingly. In contrast, higher symmetry between the user's left side and right side can result in a higher symmetry score, indicating well-balanced muscle activation between the left and right sides. The user may receive a display message recommending maintaining their form.

Part Two calculates the contribution of the user's left side and right side to overall asymmetry. This score helps the user identify which side of their body contributes more to imbalance using the following two equations:

$$Left Contribution ( \% ) = \frac{\sum \vert Symmetry Value ( Negative ) \vert}{\sum \vert Symmetry Values \vert} \times 100$$

where: Symmetry Value (Negative) is the symmetry measurement representing left-side activation (range: −100 to 100), and Σ|Symmetry Values| is the total of the absolute values across all measurements.

$$Right Contribution ( \% ) = \frac{\sum Symmetry Value ( Positive )}{\sum \vert Symmetry Values \vert} \times 100$$

where: Symmetry Value (Positive) is the symmetry measurement representing right-side activation, and Σ|Symmetry Values| is the total of the absolute values across all measurements.

The left and right contributions are then compared with each other to determine the balance between both sides for the exercise, defined as a left/right contribution score. The left/right contribution score can be displayed to the user and/or physician during the exercise and/or after the exercise is complete. The score can be categorized into multiple ranges which may also be color-coded for the display.

In one example, a left/right contribution score between, e.g., 45% to 55%, can be displayed with, e.g., a green color, indicating excellent symmetry (e.g., both sides contribute nearly equally to the overall performance). A left/right contribution between, e.g., 55% to 65%, can be displayed with, e.g., a yellow color, indicating moderate symmetry (e.g., slight domination of one side). A left/right contribution of above, e.g., 65%, can be displayed with, e.g., a red color, indicating extreme imbalance (e.g., one side heavily compensates for the other side). The left/right contribution can thus provide dynamic feedback for the user and/or patient during the exercise.

Auto-calibration technology can be built by utilizing the first (up to four) isokinetic movements of the patient to determine the normalized muscle intensity required to complete a movement. Once determined, there are a set number of repetitions of the same isokinetic movement where the calibration system will track fatigue and muscle activation for each repetition where the system according to one embodiment can increase or decrease the number of repetitions based on patient performance. The fatigue can be determined by calculating the increased number of motor neurons required to complete the isokinetic movement.

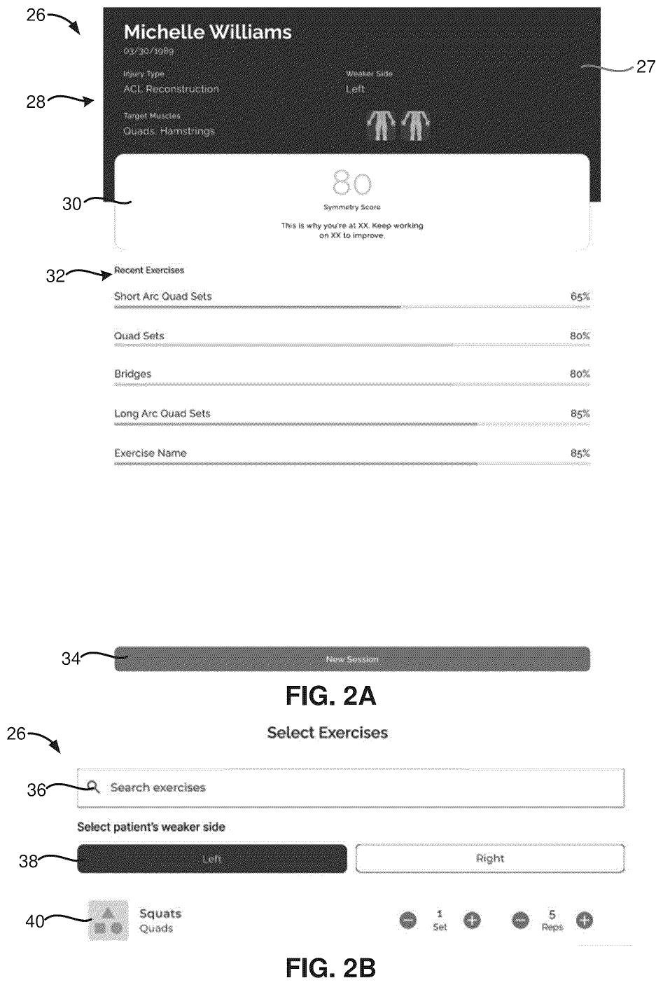

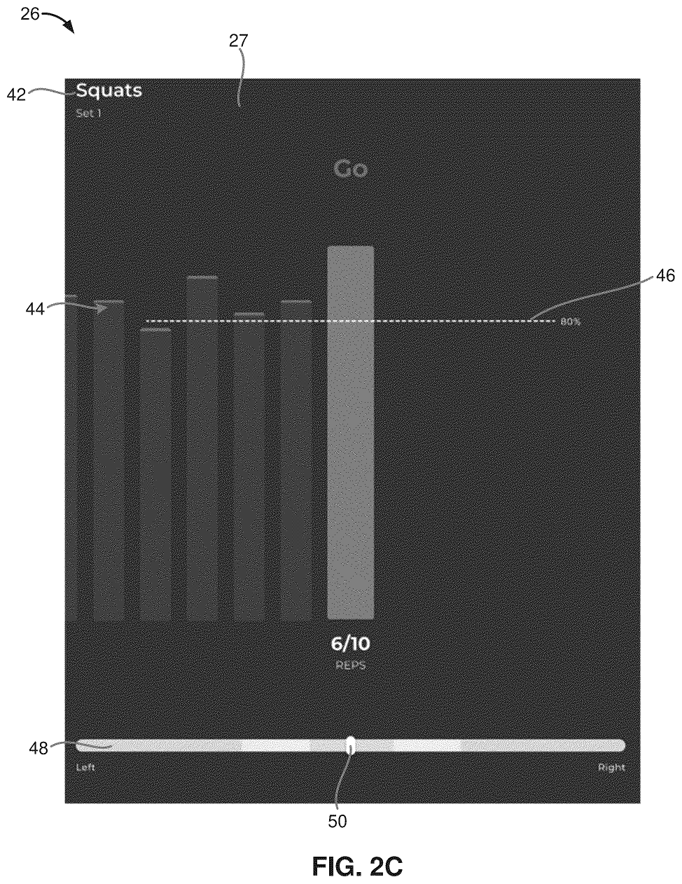

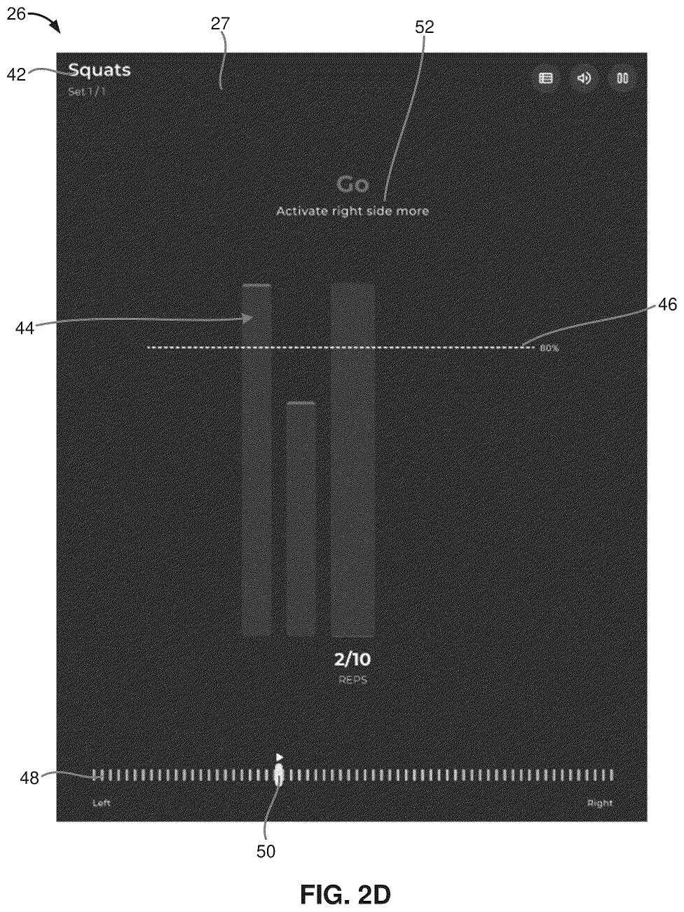

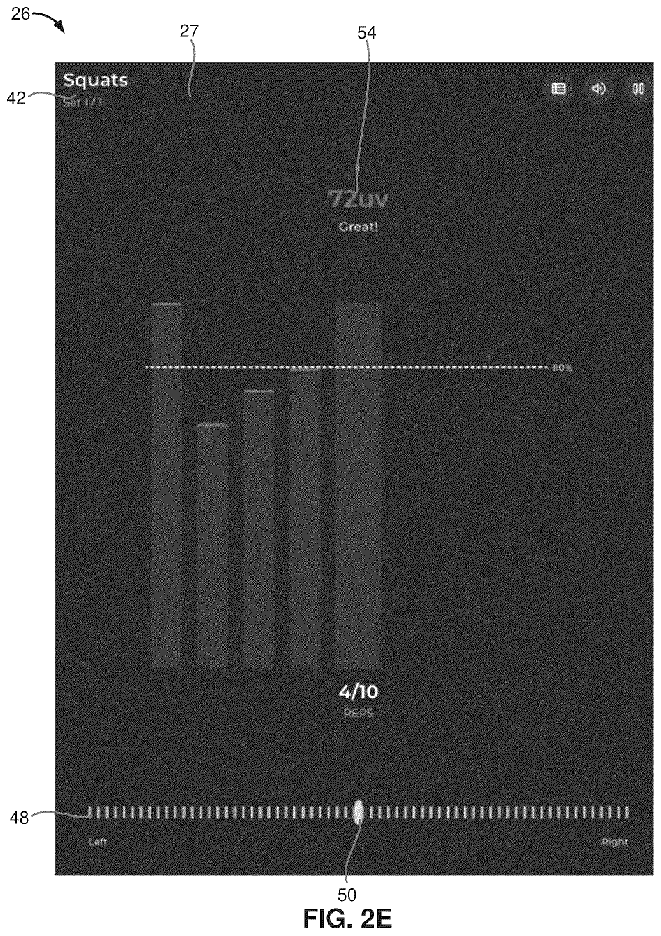

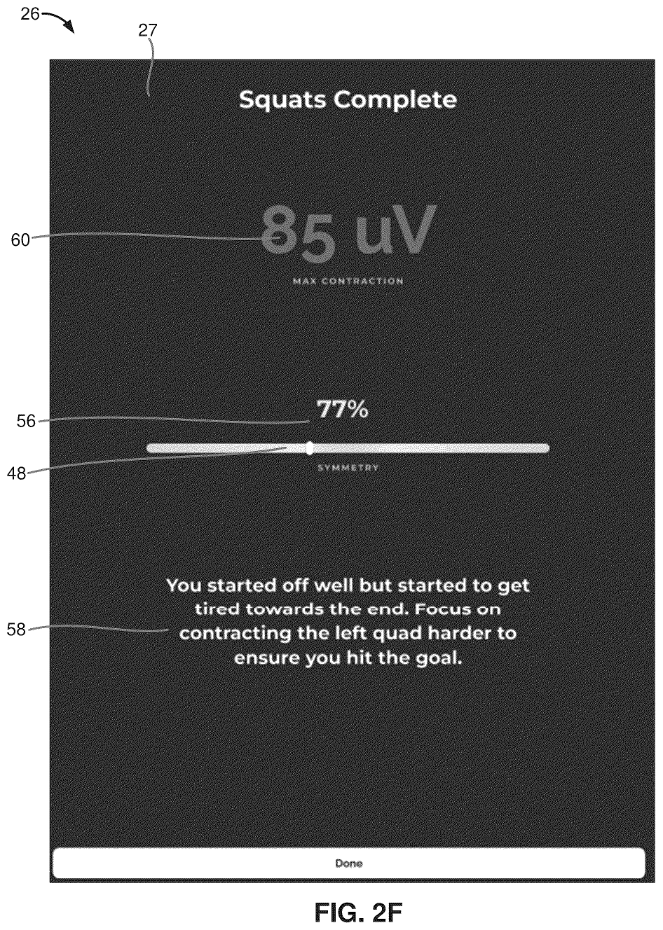

To this end, the control unit can automatically calibrate to the user with every repetition (e.g., the first repetition, the second repetition, etc.), identifying asymmetries and underperformance both during and after the exercise. Based on this data, personalized text feedback such as a prompt with respect to the calibrated goal on the display can guide patients on how to improve their exercise through balancing their muscle activation. In some instances, corrective exercises can be suggested to focus on the less dominant side of the exercise. In other instances, measures such as physical therapy or special training can be suggested to correct asymmetry. As seen in FIG. 2C, the display 27 can show a horizontal (or vertical in some instances) bar 48 with a slider 50 that illustrates to the user towards which side (e.g., left or right) the user's balance is asymmetric towards.

Part Three calculates a phase symmetry score and a standard deviation for the first, second, and final thirds of the exercise session. The exercise can be divided into three equal phases (e.g., beginning, middle, end) based on the total number of repetitions in the exercise (n). If n is not divisible by 3, some phases can have an extra repetition to ensure all data is included.

The symmetry score (Sₚₕₐₛₑ) is calculated by the equation below:

$${S}_{p h a s e} = 100 - \frac{\sum \vert {B}_{i} \vert}{n}$$

where |Bᵢ| is the absolute value of the balance bar data for each repetition and n is the number of repetitions in the phase (e.g., beginning, middle, end).

The symmetry score (phase) output by this equation can range from, e.g., 0 to 100, where 0 is complete asymmetry and 100 is perfect symmetry. This score can be displayed to the user and/or physician after the exercise is complete. The score can be categorized into multiple ranges which may also be color-coded for the display.

In one example, a symmetry score (phase) range between, e.g., 95 and 100, can be displayed with, e.g., a green color, indicating exceptional symmetry (e.g., well-balanced activation between the left and right sides of the user). A symmetry score range between, e.g., 85 and 95, can be displayed with, e.g., a yellow color, indicating minor asymmetry (e.g., a slight dominance of one side compared to the other side). A symmetry score between, e.g., 0 and 85, can be displayed with, e.g., a red color, indicating significant asymmetry (e.g., major dominance of one side which can lead to joint or muscular issues if not addressed).

In some variations, the processor can calculate the symmetry score across multiple phases of dynamic movement (e.g., initiation, peak activation, and deceleration). The process can also calculate the symmetry score by identifying variations in muscle activation patterns that influence movement precision and control.

In addition, phase variability (SDₚₕₐₛₑ), the standard deviation of the absolute values of the balance bar data in each phase, can be calculated with the following equation:

$$S {D}_{p h a s e} = \sqrt{\frac{\sum {( \vert {B}_{i} \vert - \vert \overline{B} \vert )}^{2}}{n}}$$

where |Bᵢ| is the absolute value of the balance bar data for each repetition and |B̅| is the average of the absolute values of the balance bar data in the phase.

In one example, a variability score between, e.g., 0 and 5, can be displayed with, e.g., a green color, indicating low variability and consistent movements within the exercise phase. A variability score between, e.g., 6 and 10, can be displayed with, e.g., a yellow color, indicating moderate variability and some inconsistent movements within the exercise phase. A variability score, e.g., over 10, can be displayed with, e.g., a red color, indicating high variability and many inconsistent movements within the exercise phase. The dynamic feedback can vary based on the variability score (e.g. the consistency of muscle activation) during the one or more physical movements. Accordingly, the variability score can be used to capture and analyze variability and neuromuscular control trends over time.

The phase symmetry score can be included on the display 27 so as to help patients identify when during the session asymmetries occur, allowing them to focus on improving specific phases of their exercise performance. The display can also show dynamic feedback or guidance via, e.g., text prompts or other feedback mechanisms, and can include suggestions that the user reduce, e.g., sudden shifts, improve stability, and/or focus on smoother and more controlled repetitions.

In some variations, the feedback can be a text-based summary of activation trends and methods such that the user can improve their function during the one or more exercise. In some variations, the feedback can be in the form of graphical displays (e.g., heat maps, symmetry bars) to highlight imbalances and muscle engagement patterns over time. In some variations, the feedback can be scoring metrics which provide performance-based tracking, allowing clinicians to tailor interventions more effectively.

At the end of the exercise, the display 27 can generate a report showing, e.g., symmetry scores across multiple sessions. In some instances, the symmetry score can be processed by using, e.g., one, two, or all three of the parts mentioned above.

The dynamic feedback displayed to the user can be based on the symmetry score of each of the plurality of phases of the one or more physical movements. For example, in one variation, excellent symmetry (e.g., a symmetry score of 90 to 100) in each of the three phases (beginning, middle and end) can output a feedback message: “You maintained great form and balance throughout, finishing strong with consistency. Keep up the great work!” In another example, significant imbalance (e.g., a symmetry score of 0 to 69) in each of the three phases (beginning, middle and end) can output a feedback message You started with significant imbalances, and it's important to work on better balance. In the middle, significant imbalances were observed, and control needs to improve. “You finished with severe imbalances, likely due to fatigue, which should be addressed. Focus on sustaining effort in the middle phase to improve performance. Work on starting exercises with better balance and control. Pay attention to controlling fatigue as you finish.” The dynamic feedback can range between these two examples based on the symmetry score in each phase.

In other variations, the symmetry score can be based on detected stability and endurance trends to measure fatigue impact on performance.

FIGS. 2A to 2F illustrate one variation of the display 27 shown to the user or the physician. FIG. 2A illustrates a user interface 26 on the display that comprises user details 28 (e.g., date of birth, injury, target muscles, weaker side) and an overall symmetry score 30 and symmetry scores 32 for previous exercises. The display can also comprise a button 34 for initiating a new exercise session.

FIG. 2B illustrates a user interface 26 on the display 27 in which the user or physician can prepare a new exercise session. This user interface 26 can comprise a search bar 36 for entering the desired exercise, a button 38 for selecting the user's weaker side (if necessary), and an interface 40 to enter the amount of sets and repetitions are to be performed in the exercise.

FIG. 2C illustrates a user interface 26 on the display 27 for view during the exercise. The user interface 26 can comprise a label 42 for the exercise. The user interface 26 can comprise a vertical status bar 44 or graphical display for the symmetry score during the exercise to be displayed along with a horizontal threshold bar 46 set at a value (e.g., 80%) determined by the user. The vertical status bar 44 can display the symmetry score which is calculated by the processor for each repetition and can increase in height based on the symmetry score for each repetition. The vertical status bar 44 can also display which number repetition (e.g., 6/10) the user is currently performing.

The user interface 26 can also comprise a horizontal status bar 48 that displays the left/right contribution score calculated by the processor. The horizontal status bar 48 can be marked with “left” and “right” markers and can comprise a slider 50 that indicates the left/right contribution for each repetition. In one example, the slider 50 can be in the middle of the horizontal status bar 48, indicating a left/right contribution of about 50%, indicating excellent symmetry between the user's left and right sides. In other examples, the slider 50 can indicate asymmetry by sliding to the left or the right side respective to the relative strength of the corresponding side. The horizontal status bar 48 can also be color-coded (e.g., green, yellow, orange red) to further indicate their symmetry in real-time to the user indicating the user is, e.g., well balanced, has minor asymmetry, or major asymmetry in their movement.

In one example, as seen in FIG. 2D, asymmetry is detected towards the left side of the user. Accordingly, the slider 50 of the horizontal status bar can move towards the left side of the bar providing a visual indicator to the user of their asymmetric movement and the degree of their asymmetry. The display can also provide a word prompt 52 to the user (e.g., “activate right side more”) or other visual and/or auditory prompt in real-time based on the measured muscle activation and corresponding symmetry score.

As seen in FIG. 2E, the user interface 26 can also display the user's measured electrical activity of the muscles (e.g., in u V (microvolts) 54) for a corresponding repetition. This feedback can inform the user of the energy exerted in a specific repetition.

FIG. 2F illustrates a user interface 26 on the display 27 that is shown at the end of the exercise. The display bar can show both an overall symmetry score 56 and the horizontal status bar 48 in the direction asymmetry has occurred during the exercise. The user interface 26 can also comprise a word prompt 58 or other visual, haptic, and/or auditory prompt to the user based on the symmetry score (phase). The word prompt 58 (or other visual, haptic, and/or auditory feedback) can comprise feedback to the user stating in which phase the user experienced fatigue and/or which side of the body may need to be focused on during the next exercise. The display can also show a maximum amount of energy exerted during u V 60 the exercise, e.g., measuring the electrical activity of the muscles.

In another variation, the system uses a surface EMG and TENS technology to automatically calibrate goals based on the intensity of muscle firing and live fatigue score to build strength during therapy. The data can be tracked historically to view improvements in strength, fatigue, and balance across in-clinic and at-home settings.

In another variation, the system provides an adaptive rehabilitation training machine using surface electromyography (EMG), including: a surface EMG device configured to capture EMG signals from a muscle group of a user and to establish a calibrated goal based on the captured EMG signals during an auto-calibration phase and isokinetic movement; a repetition detection algorithm integrated into the surface EMG device for automatically detecting repetitions of exercises performed by the user during subsequent exercise sessions; a feedback mechanism integrated into the surface EMG device, including auditory, visual, and haptic feedback, to notify the user when the calibrated goal is achieved during an exercise session; a fatigue monitoring module configured to monitor fatigue levels of the user based on changes in the surface EMG signals during the exercise session; a processor configured to adjust the calibrated goal based on the detected fatigue levels and to provide feedback to the user after each exercise session, including a score determined based on the success of repetitions in meeting the calibrated goal and the intensity of the exercises performed; and an electrical impulse device that stimulates nerve and muscle contraction after the auto-calibration.

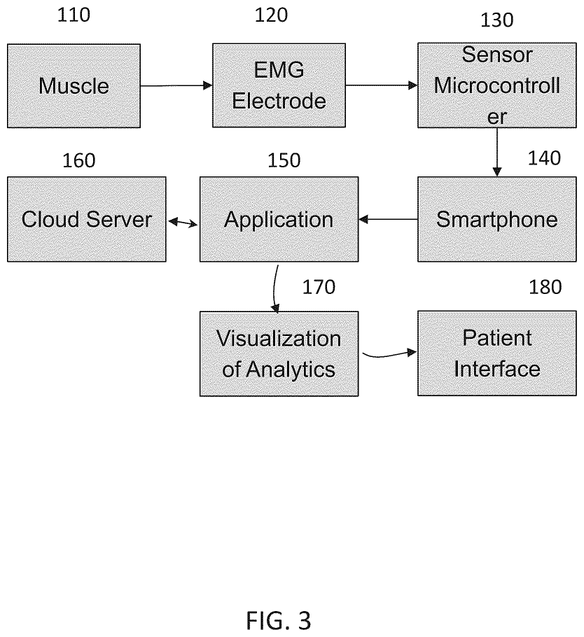

FIG. 3 illustrates a surface EMG flowchart showing how the elements are connected according to one embodiment. As shown in FIG. 3, one or more EMG electrodes 120 are attached to the surface of a muscle or muscle group 110. The sensor microcontroller 130 receives the EMG signals sensed via the EMG electrode 120 through a wired or wireless connection to the microcontroller. A smart phone or smart device 140 or the sensor microcontroller 130 receives the data and processes the data with a rehabilitation training software application 150. The processed results can be uploaded to a remotely located storage location such as a cloud server 160 or other server for storage. The application or algorithm 150 generates visualization of analytics 170, and provides feedback to the user via the patient interface 180.

According to one embodiment, the rehabilitation training machine may include a TENS device. The TENS device provides electrical pulses to the muscle. Electrical pulses that stimulate nerves and contract muscles provide the following benefits: (1) increase muscle performance and strength, (2) help muscle recovery, and (3) reduce muscle pain.

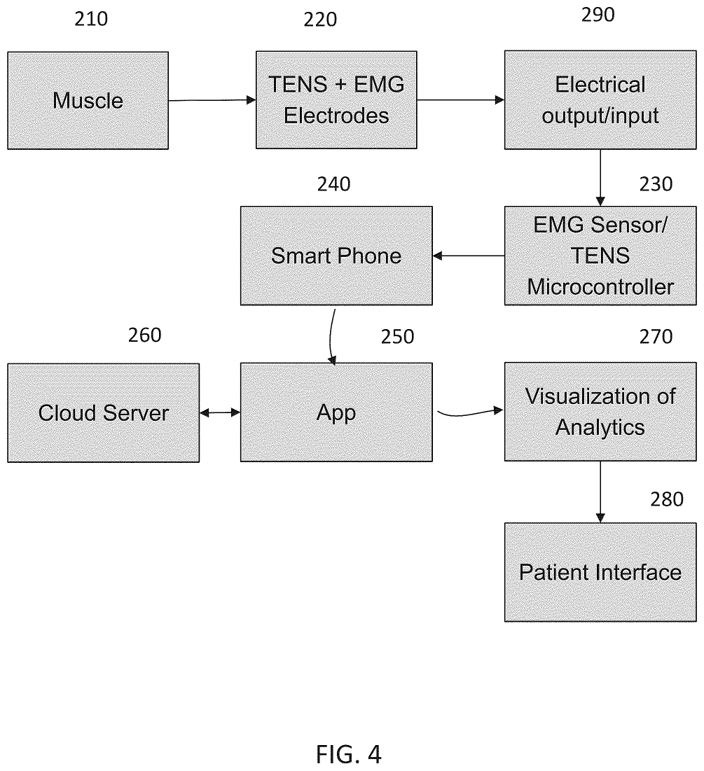

FIG. 4 illustrates a TENS plus EMG flowchart showing how the elements are connected according to one embodiment. As shown in FIG. 4, one or more TENS and EMG electrodes 220 are attached to the surface of a muscle or muscle group 210. The EMG sensor/TENS microcontroller 230 receives signals from the muscle attached to the EMG electrodes 220 via an electrical output/input 290 and provides an electrical pulse to the muscle attached to the TENS electrodes via the electrical output/input 290. A smart phone or smart device 240 or the sensor microcontroller 230 receives the data and processes the data with a rehabilitation training software application 250. The processed results can be uploaded to a remotely located storage location such as a cloud server 260 or other server for storage. The application or algorithm 250 generates visualization of analytics 270, and provides feedback to the user via the patient interface 280.

According to one embodiment, the rehabilitation training machine may include a remote monitoring capability. In one embodiment, the calibrated system can be used to measure the ideal number of sets and repetition of an exercise and provide this as a home exercise program that the therapists can track patient activity remotely.

In one embodiment, the system performs specific muscle analysis, including muscle strength and muscle fatigue.

Strength is measured through muscle intensity by calculating the number of motor neurons required to complete a specific movement. This takes the peak amplitude of EMG data during an isokinetic movement.

Fatigue is measured through the human body's natural response to increase the number of motor neurons from nearby muscles to help complete an isokinetic movement. This indicates the targeted muscle has reached a point of failure where the muscle requires additional energy. The system according an embodiment measures fatigue as a way of building patient strength and endurance and provides real-time calibration to limit risk of injury and improve strength.

It is contemplated that other applications may use the disclosed technology by taking raw data from EMG sensors to calibrate specified exercise goals based on muscle intensity and real-time fatigue. This calibration can be used for providing automated TENS electrical impulses, viewing historical improvements for patients (primarily regarding strength, fatigue, and balance), and also building patient-specified home exercise programs. A combination of these outputs is derived from an automated calibration system as discussed herein.

Case studies have shown that the use of sEMG BF in an adaptive rehabilitation training machine according to an embodiment helps patients increase their muscle strength and improve their range of movement. The improvement in muscle control and strength has been shown to be significant over traditional therapy systems.

While the present disclosure describes at some length and with some particularity with respect to the several described embodiments, it is not intended that it should be limited to any such particulars or embodiments or any particular embodiment, but it is to be construed so as to provide the broadest possible interpretation in view of the related art and, therefore, to effectively encompass various embodiments herein. Furthermore, the foregoing describes various embodiments foreseen by the inventor for which an enabling description was available, notwithstanding those modifications of the disclosure, not presently foreseen, may nonetheless represent equivalents thereto.

Each of the individual variations described and illustrated herein has discrete components and features which may be readily separated from or combined with the features of any of the other variations. Modifications may be made to adapt a particular situation, material, composition of matter, process, process act(s) or step(s) to the objective(s), spirit or scope of the present invention.

Methods recited herein may be carried out in any order of the recited events which is logically possible, as well as the recited order of events. Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

What is claimed is:

1. A system for rehabilitation training for a user, the system comprising:
a sensor configured to capture one or more EMG signals from a muscle group of the user when performing one or more physical movements;
a processor configured to analyze the one or more EMG signals, wherein the processor is configured to determine a symmetry score which is indicative of a relative symmetry of the muscle group during the one or more physical movements and is based on a comparison of muscle activation in the muscle group, wherein the processor is configured to establish an adaptive target goal based on the muscle activation in the muscle group; and
a display configured to provide guidance to the user during the one or more physical movements, wherein the guidance comprises a dynamic feedback for the user based on the symmetry score and is auditory, haptic, and/or visual to guide the user on a target muscle activation level.

2. The system of claim 1, wherein the processor is configured to calculate the symmetry score across one or more phases of muscle activation.

3. The system of claim 1, wherein the processor is configured to identify variations in one or more muscle activation patterns that influence movement precision and control.

4. The system of claim 1, wherein the adaptive target goal established by the processor is based on a predetermined threshold relating to the muscle activation, wherein the predetermined threshold is a percentage of a highest value of the one or more EMG signals.

5. The system of claim 1, wherein the processor calculates the symmetry score by taking an average asymmetry of muscle activation via the one or more EMG signals between a first lateral side of the user and a second lateral side of the user.

6. The system of claim 1, wherein the processor calculates the symmetry score in real-time during the one or more physical movements by comparing muscle activation via the one or more EMG signals between a first lateral side of the user and a second lateral side of the user.

7. The system of claim 1, wherein the processor calculates the symmetry score in a plurality of phases of the one or more physical movements.

8. The system of claim 1, wherein the symmetry score comprises a consistency of muscle activation via the one or more EMG signals during the one or more physical movements, wherein the processor is configured to assess the consistency by capturing variability and neuromuscular control trends of the muscle group over time.

9. The system of claim 1, wherein the symmetry score comprises stability and endurance trends to measure fatigue of the muscle group during the one or more physical movements.

10. A method for measuring rehabilitative exercises of a patient, the method comprising:
applying a sensor to a user;
capturing one or more EMG signals with the sensor, wherein the one or more EMG signals are derived from a muscle group of a user when performing one or more physical movements;
analyzing the one or more EMG signals with a processor;
determining a symmetry score with the processor, wherein the symmetry score which is indicative of a relative symmetry of the muscle group during the one or more physical movements and is based on a comparison of muscle activation in the muscle group, wherein the processor is configured to establish an adaptive target goal based on the muscle activation in the muscle group; and
providing guidance to the user with a display during the one or more physical movements, wherein the guidance comprises a dynamic feedback for the user based on the symmetry score and is auditory, haptic, and/or visual to guide the user on a target muscle activation level.

11. The method of claim 10, wherein the processor is configured to calculate the symmetry score across one or more phases of muscle activation.

12. The method of claim 10, wherein the processor is configured to identify variations in one or more muscle activation patterns that influence movement precision and control.

13. The method of claim 10, wherein the adaptive target goal established by the processor is based on a predetermined threshold relating to the muscle activation, wherein the predetermined threshold is a percentage of a highest value of the one or more EMG signals.

14. The method of claim 10, further comprising calculating the symmetry score by taking an average asymmetry of muscle activation via the one or more EMG signals between a first lateral side of the user and a second lateral side of the user.

15. The method of claim 10, further comprising calculating the symmetry score in real-time during the one or more physical movements by comparing muscle activation via the one or more EMG signals between a first lateral side of the user and a second lateral side of the user.

16. The method of claim 10, further comprising calculating the symmetry score in a plurality of phases of the one or more physical movements.

17. The method of claim 16, wherein the dynamic feedback varies based on the symmetry score of each of the plurality of phases of the one or more physical movements, wherein the dynamic feedback comprises a text, a numerical score, or a graphical display.

18. The method of claim 10, wherein the symmetry score comprises a consistency of muscle activation via the one or more EMG signals during the one or more physical movements, wherein the processor is configured to assess the consistency by capturing variability and neuromuscular control trends of the muscle group over time.

19. The method of claim 18, wherein the dynamic feedback varies based on the consistency of muscle activation during the one or more physical movements, wherein the dynamic feedback comprises a text, a numerical score, or a graphical display.

20. A method for measuring rehabilitative exercises of a patient, the method comprising:
applying a sensor to a user;
capturing one or more EMG signals with the sensor, wherein the one or more EMG signals are derived from a muscle group of a user when performing one or more physical movements;
analyzing the one or more EMG signals with a processor;
determining a calibrated goal based on a predetermined threshold relating to muscle activation of the muscle group, wherein the calibrated goal is adaptive based on changes in muscle activation; and
providing guidance to the user with a display during the one or more physical movements, wherein the guidance comprises a dynamic feedback for the user based on the calibrated goal, wherein the guidance is auditory, haptic, and/or visual to guide the user on a target muscle activation level.
